(19) United States
(12) Patent Application Publication
(10) Pub. No.: US 2009/0291132 A1
(43) Pub. Date: Nov. 26, 2009

(54) Enhanced delivery of antifungal agents

(76) Inventors: Brian Charles Keller, Antioch, CA (US); Nian Wu, North Brunswick, NJ (US)

Correspondence Address: Lee Pederson, 712 East Main Street, Sleepy Eye, MN, 56085, US

(21) Appl. No.: 12/454,387

(22) Filed: May 15, 2009

Related U.S. Application Data
(63) Continuation-in-part of application No. 12/006,820, filed on Jan. 4, 2008.
(60) Provisional application No. 61/128,011, filed on May 16, 2008.

(51) Int. Cl. A61K 9/127; A61K 31/496

(52) U.S. Cl. 424/450; 514/254.07

(57) ABSTRACT

Tetrahydrofuran antibiotics formulated with certain diacylglycerol-poltethyleneglycol (DAG-PEG) lipids have increased water solubility and bioavailability. PEG-12 GDO, PEG-12 GDLO, PEG-12 GDM and PEG-12 GDP are particularly suitable for forming liposomes that incorporate tetrahydrofurans in the bilayer.

### RELATED APPLICATIONS

This application claims priority as a continuation-in-part application to co-pending U.S. patent application Ser. No. 12/006,820, entitled “Enhanced Delivery of Antifungal Agents” and filed Jan. 4, 2008. This application also claims priority to provisional U.S. Patent Application 61/128,011, entitled “Enhanced Delivery of Antifungal Agents” and filed May 16, 2008.

### FIELD OF THE INVENTION

This invention relates to drug delivery, particularly to delivery of tetrahydrofuran antifungal agents by microencapsulation and liposomal formulation.

### BACKGROUND OF INVENTION

Tetrahydrofuran antibiotics are widely used as antifungal agents. They include the drug ketoconazole and are described in U.S. Pat. No. 5,039,676. Newer tetrahydrofurans have been developed that are more effective and less toxic than ketoconazole. They are described in U.S. Pat. No. 5,661,151. The newer tetrahydrofurans include posaconazole and itraconazole.

Current disadvantages of tetrahydrofurans include poor water solubility and limited bioavailability. Therefore, it is desirable to develop formulations which address these limitations.

### BRIEF DESCRIPTION OF THE INVENTION

Tetrahydrofuran antibiotics formulated with certain diacylglycerol-polytethyleneglycol (DAG-PEG) lipids have increased water solubility and bioavailability. PEG-12 GDO, PEG-12 GDLO, PEG-12 GDP and PEG-12 GDM are particularly suitable for forming liposomes that incorporate tetrahydrofurans in the bilayer.

### BRIEF DESCRIPTION OF THE FIGURES

FIG. 1 shows comparison between blood concentrations of various posaconazole formulations administered intravenously.

FIG. 2 shows a comparison between blood concentrations of a commercial posaconazole product and a DAG-PEG formulation of posaconazole administered orally.

FIG. 3 shows a comparison between a DAG-PEG lipid formulation of itraconazole and an aqueous solution of itraconazole containing 5% of dimethyl sulfoxide and 10% of Cremophor after IV administration.

FIG. 4 shows a comparison between oral administrations of DAG-PEG lipid formulation of itraconazole and an aqueous solution of itraconazole containing 5% of dimethyl sulfoxide and 10% of Cremophor.

### DETAILED DESCRIPTION OF PREFERRED EMBODIMENTS

“Diacylglycerol-polyethyleneglycol (DAG-PEG)” refers to a lipid with a three-carbon-chain backbone and having acyl groups attached to two of the three carbons and a polyethylene chain attached to the other carbon. The acyl and polyethylene glycol chains may be attached to the backbone by a variety of chemical linkages, including but not limited to, ester and ether bonds. Linkers may be provided between the backbone and the chains. The chains may be attached at any position of the backbone.

U.S. Pat. No. 6,610,322, which is hereby incorporated by reference, teaches the spontaneous formation of liposomes when certain lipids are added to aqueous solutions. Table 1 shows lipids described in that patent.

TABLE 1

| | | Packing |
|---|---|---|
| | | parameters |
| | | favorable for |
| | | liposome |
| PEG-lipid | melting temperature | formation |
| PEG-23 GDL | Fluid at 25 degrees C. | No |
| PEG-12 GDLO | Fluid at 25 degrees C. | Yes |
| PEG-12 GDO | Fluid at 25 degrees C. | Yes |
| PEG-23 GDO | Fluid at 25 degrees C. | No |
| PEG-43 GDO | 36.3 | No |
| PEG-12 GDM | Fluid at 25 degrees C. | Yes |
| PEG-23 GDM | Fluid at 25 degrees C. | No |
| PEG-45 GDM | 33.2 | No |
| PEG-12 GDP | Semisolid at 25 degrees C. | Yes |
| PEG-23 GDP | 31.2 | Yes |
| PEG-45 GDP | 41.8 | No |
| PEG-12 GDS | 40.0 | Yes |
| PEG-23 GDS | 39.8 | Yes |
| PEG-45 GDS | 40.8 | No |

In the table PEG-23 GDL refers to glycerol dilaurate having a 23 subunit PEG chain. Similarly, GDO refers to glycerol dioleates. GDM refers to glycerol dimyristates, GDP refers to glycerol dipalmitates and GDS refers to glycerol distearates.

Table 1 includes two lipids that were not described in particular in U.S. Pat. No. 6,610,322. PEG-12 GDLO refers to glycerol dilinoleate having a 12 subunit PEG chain. PEG-12 GDP refers to glycerol dipalmitate having a 12 subunit PEG chain.

PEG-12 GDP is semisolid at 25 degrees C., indicating that most of the molecules are in the disordered liquid crystalline phase and not in the ordered gel phase. Also, PEG-12 GDP readily mixes with water and forms lipsomes at 25 degrees C. Therefore, for the purposes of this patent PEG-12 GDP is considered to have a melting temperature below about 25 degrees C.

Lipid packing parameters determine whether a lipid or combination of lipids are capable of forming liposomes. Because liposomes include one or more curved lipid bilayers, spacial configurations of individual lipid molecules must be such to allow the lipids to pack together correctly in order for liposomes to form. If the hydrophilic head groups are too large in relation to the volume of the lipophilic tails, the radius of curvature will be too small to encapsulate an aqueous space and micelles will form. If the hydrophilic head groups are too small in relation to the volume of the lipophilic tails, the radius of curvature will be too large to allow the bilayer to close back upon itself and form a sphere.

Liposome forming DAG-PEG lipids are preferred for drug delivery because they form particles of uniform size. When used for delivery of lipophilic drugs, as in the present invention, they provide a maximum of uniform lipophilic environment for the drug to partition into. Because the liposomes from a stable suspension in aqueous environments, they allow for increased bioavailability and improved pharmacokinetics of such drugs.

DAG-PEG lipids with favorable packing parameters spontaneously form liposomes in aqueous environments above the melting temperature of the lipids. For example, with a melting point of 31.2 degrees C., PEG-23 GDP does not spontaneously form liposomes at room temperature. However, at temperatures above 31.2 degrees PEG-23 GDP will spontaneously form liposomes. Once liposomes are formed at higher temperatures, the liposomes will be stable even if the liposome suspension is cooled to below 31.2. With a melting temperature of about 40 degrees C. and favorable packing parameters, PEG-12 GDS behaves similarly. DAG-PEG lipids with packing parameters outside the favorable range will not spontaneously form liposomes no matter the temperature.

DAG-PEG lipids are typically not pure. For example, a batch of PEG-23 GDP will contain other DAG-PEG species having slightly different acyl chains and slightly different PEG chains. However, the properties of the batch will closely approximate a theoretically pure batch, because species with longer acyl chains will offset those with shorter acyl chains. Also species with longer PEG chains will offset those with shorter PEG chains.

It has been discovered that formulating tetrahydrofurans with DAG-PEG lipids into liposomes may result in a different outcome than forming liposomes with pure lipids. When either PEG-23 GDP, PEG-12 GDS or PEG-23 GDS is used with tetrahydrofurans, liposomes are formed when the temperature is above the melting temperature of the lipid. However, when these liposomes are cooled below the melting temperature of the lipids the liposome suspensions are not stable. Instead, they gel or precipitate (see tables 2-5). Since it is impractical to maintain the suspensions at elevated temperatures, these DAG-PEGs are not suitable for certain tetrahydrofuran/liposome formulations.

Tetrahydrofuran antibiotics are lipophilic and have low solubility in aqueous solutions. Newer tetrahydrofuran antibiotics such as posaconazole are more lipophilic than earlier ones like ketoconazole. It is expected that these drugs will overwhelmingly partition into the lipid bilayer, and that very little drug will remain in the aqueous phase. The biophysical cause of the effect described in the paragraph above is not yet clear, but it seems that the tetrahydrofurans disrupt the bilayer at temperatures below the melting temperature of the lipids. This effect is unexpected and has not previously been observed.

Combinations of lipids may be used in the present invention. As described in U.S. Pat. No. 6,610,322, combinations of lipids will have predictable physical properties such as melting temperature and packing parameters.

The tetrahydrofuran liposomes of the present invention are useful in several ways. They may be formulated into solutions for topical application. More importantly, they may be used internally to prevent and treat serious fungal infections. For internal use, they may be administered either orally or intravenously. Capsules and solutions are two possible formulations for oral administration. When intended for IV use, there are also several possible formulations. The liposomes may be manufactured and distributed as a liposome suspension. They may be manufactured as a powder containing the drug, lipid, and a sugar such as sucrose. Also, they may be manufactured as dehydrated liposomes. The powder may be stored until needed. Then upon addition of water the powder will either form or reconstitute liposomes.

The liposomal formulations of the present invention are advantageous because they offer increased bioavailability and potentially reduced toxicity for tetrahydrofuran delivery. Laboratory studies have shown increased bioavailability over non-liposome preparations for both oral and IV administration. Additionally, the liposomes of the present invention have been shown to be superior to liposome formulations using DOPC (diolealphosphatydilcholine).

The formulations described here have been described as liposome suspensions, but the formulations may include other types of microparticles. The formulations described in the examples (excluding example 10) were observed using electromicroscopy. Small aggregates (˜10 nm) and various sizes of multilamellar vesicles (100 to 500 nm) were observed, which showed that the solubilization of drugs was achieved by the lipid encapsulation.

In one aspect the invention is a pharmaceutical composition comprising a tetrahydrofuran drug, and a DAG-PEG lipid having a melting temperature below about 25 degrees C. and having packing parameters favorable for the formation of liposomes. A combination of DAG-PEG lipids may be used. The drug may be selected from the group consisting of posaconazole, itraconazole and equaconazole. The DAG-PEG lipid may be selected from the group consisting of PEG-12 GDO, PEG-12 GDLO, PEG-12 GDM and PEG-12 GDP. The drug to lipid ratio is preferably greater than about 1 to 20, and more preferably greater than about 1 to 5.

The composition may comprise a sugar and be in a dehydrated form for later addition with an aqueous solution. The composition may be a liposome suspension. The composition may comprise a combination of DAG-PEGs. The drug concentration is preferably between about 1 to 50 mg/ml. The composition may include solid excipients and be in the form of a capsule for oral administration.

In another aspect the invention is method of making a pharmaceutical formulation comprising selecting a tetrahydrofuran drug; selecting a diacyl PEG lipid having a melting temperature below about 25 degrees C. and having packing parameters favorable for the formation of liposomes; and combining the drug with the lipid in an aqueous solution. The drug may be selected from the group consisting of posaconazole, itraconazole and equaconazole. The lipid may be selected from the group consisting of PEG-12 GDO, PEG-12 GDLO, PEG-12 GDM and PEG-12 GDP. The final drug concentration is preferably between about 1 to 50 mg/ml. The final drug to lipid ratio is preferably greater than about 1 to 20, and more preferably greater than about 1 to 5.

In another aspect the invention is method of treating or preventing a fungal infection comprising selecting a tetrahydrofuran drug; selecting a diacyl PEG lipid having a melting temperature below about 25 degrees C. and having packing parameters favorable for the formation of liposomes; combining the drug with the lipid in an aqueous solution; and administering the solution. The drug may be selected from the group consisting of posaconazole, itraconazole and equaconazole. The lipid may be selected from the group consisting of PEG-12 GDO, PEG-12 GDLO, PEG-12 GDM and PEG-12 GDP. The final drug concentration is preferably between about 1 to 50 mg/ml. The final drug to lipid ration is preferably greater than about 1 to 20, and more preferably greater than about 1 to 5. The administration may be oral, topical or intravenous.

The gelation effect described in paragraphs 17 and 18 above can be exploited as a separate invention. Sometimes gelation of a tetrahydrofuran/lipid composition is desired, such as in preparation of a topical cream. In such cases, PEG-12 GDS, PEG-23 GDS, and PEG 23 GDP may be used separately or in combination to prepare topical tetrahydrofuran creams. In this aspect, the separate invention is a pharmaceutical cream comprising a tetrahydrofuran drug, and a DAG-PEG lipid having a melting temperature above about 25 degrees C. and having packing parameters favorable for the formation of liposomes. The drug may be selected from the group consisting of ketoconazole, posaconazole, itraconazole and equaconazole. The DAG-PEG lipid may be selected from the group consisting of PEG-12 GDS, PEG-23 GDS and PEG-23 GDP.

Solutions may be applied topically, also. If topical application of a solution is desired, lipids that do not exhibit the gelation effect are selected.

In another aspect, the invention includes a new tetrahydrofuran drug that is referred to herein as equaconazole. Equacoazole has the following structure(s).

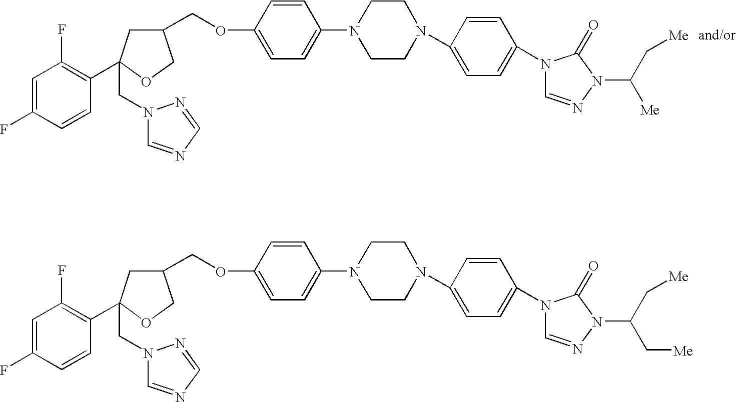

While preferred embodiments of the present invention have been described, those skilled in the art will recognize that other and further changes and modifications can be made without departing from the spirit of the invention, and all such changes and modifications should be understood to fall within the scope of the invention.

### Example 1

### Itraconazole-Lipid Formulations

Itraconazole was combined in an aqueous solution with DAG-PEG lipids. A variety of drug-to-lipid ratios were tested, as well as a variety of formation temperatures. After mixing, formulations were allowed to stand at room temperature. The results are shown in Table 2.

TABLE 2

| (a) | | | | | | |
|---|---|---|---|---|---|---|
| Lipid w/ Itraconazole | Lipid Conc | Drug Solubility¹ | | | | |
| (30 mg drug/mL) | (mg/mL) | 25° C. | 35° C. | 45° C. | 60° C. | Comment |
| PEG-23 GDL | 150 | −/+ | + | + | ++ | |
| PEG-12 GDO | 150 | + | + | ++ | +++ | |
| PEG-12 GDLO | 150 | + | + | ++ | +++ | |
| PEG-23 GDO | 200 | + | + | + | ++ | |
| PEG-43 GDO | 200 | − | −/+ | + | ++ | S |
| PEG-12 GDM | 150 | + | + | ++ | +++ | |
| PEG-23 GDM | 200 | + | + | + | ++ | |
| PEG-45 GDM | 200 | − | −/+ | + | + | S |
| PEG-12 GDP | 200 | −/+ | + | ++ | ++ | |
| PEG-23 GDP | 200 | − | −/+ | + | + | S |
| PEG-45 GDP | 200 | − | −/+ | + | + | S |
| PEG-12 GDS | 200 | − | −/+ | + | + | S |
| PEG-23 GDS | 200 | − | + | + | + | S |
| PEG-45 GDS | 200 | − | − | + | + | S |

| (b) | Drug Solubilized @ 40° C. | | | | | |
|---|---|---|---|---|---|---|
| Lipid w/ Itraconazole | | | L/D | L/D | L/D ratio = | |
| (30 mg drug/mL) | L/D ratio = 1 | L/D ratio = 3 | Ratio = 5 | ratio = 10 | 20 | Comment |
| PEG-23 GDL | − | − | + | + | + | |
| PEG-12 GDO | − | ++ | + | ++ | ++ | |
| PEG-12 GDLO | − | ++ | + | ++ | +++ | |
| PEG-23 GDO | − | − | + | + | + | |
| PEG-43 GDO | − | − | −/+ | + | + | S |
| PEG-12 GDM | − | ++ | + | + | + | |
| PEG-23 GDM | − | − | + | + | + | |
| PEG-45 GDM | − | − | −/+ | + | + | S |
| PEG-12 GDP | − | + | + | ++ | ++ | |
| PEG-23 GDP | − | − | + | + | + | S |
| PEG-45 GDP | − | − | + | + | + | S |
| PEG-12 GDS | − | − | + | + | + | S |
| PEG-23 GDS | − | − | + | + | + | S |
| PEG-45 GDS | − | − | −/+ | + | + | S |

In Tables 2, 3, 4 and 5, “−” means not soluble; “−/+” means partially soluble; “+” soluble, “++” very soluble, “+++” most soluble. “S” indicates gelation or precipitation when the formulation was held at 25° C.

### Example 2

### Posaconazole-Lipid Formulations

Posaconazole was combined in an aqueous solution with DAG-PEG lipids. A variety of drug-to-lipid ratios were tested, as well as a variety of formation temperatures. After mixing, formulations were allowed to stand at room temperature. The results are shown in Table 3.

TABLE 3

| (a) | | | | | | |
|---|---|---|---|---|---|---|
| Lipid w/ Posaconazole | Lipid Conc | Drug Solubility¹ | | | | |
| (30 mg drug/mL) | (mg/mL) | 25° C. | 35° C. | 45° C. | 60° C. | Comment |
| PEG-23 GDL | 150 | + | + | ++ | ++ | |
| PEG-12 GDO | 90 | + | ++ | ++ | +++ | |
| PEG-12 GDLO | 150 | + | + | ++ | +++ | |
| PEG-23 GDO | 120 | + | ++ | ++ | ++ | |
| PEG-43 GDO | 120 | − | −/+ | + | ++ | S |
| PEG-12 GDM | 90 | + | ++ | ++ | +++ | |
| PEG-23 GDM | 150 | + | + | ++ | ++ | |
| PEG-45 GDM | 150 | − | −/+ | + | + | S |
| PEG-12 GDP | 150 | −/+ | + | ++ | ++ | |
| PEG-23 GDP | 120 | − | −/+ | + | + | S |
| PEG-45 GDP | 150 | − | −/+ | + | + | S |
| PEG-12 GDS | 90 | − | −/+ | + | + | S |
| PEG-23 GDS | 120 | − | + | + | + | S |
| PEG-45 GDS | 120 | − | − | −/+ | + | S |

| (b) | Drug Solubilized @ 40° C. | | | | | |
|---|---|---|---|---|---|---|
| Lipid w/ Posaconazole | | | L/D | L/D | LD ratio = | |
| (30 mg drug/mL) | L/D ratio = 1 | L/D ratio = 3 | ratio = 5 | ratio = 10 | 20 | Comment |
| PEG-23 GDL | − | − | + | + | + | |
| PEG-12 GDO | −/+ | + | ++ | ++ | ++ | |
| PEG-12 GDLO | −/+ | + | + | ++ | +++ | |
| PEG-23 GDO | − | − | + | + | + | |
| PEG-43 GDO | − | − | −/+ | + | + | S |
| PEG-12 GDM | −/+ | + | ++ | ++ | ++ | |
| PEG-23 GDM | − | − | + | + | + | |
| PEG-45 GDM | − | − | −/+ | + | + | S |
| PEG-12 GDP | − | −/+ | + | ++ | ++ | |
| PEG-23 GDP | − | − | + | + | + | S |
| PEG-45 GDP | − | − | + | + | + | S |
| PEG-12 GDS | − | − | + | + | + | S |
| PEG-23 GDS | − | − | + | + | + | S |
| PEG-45 GDS | − | − | −/+ | + | + | S |

### Example 3

### Equaconazole-Lipid Formulations

Equaconazole was combined in an aqueous solution with DAG-PEG lipids. A variety of drug-to-lipid ratios were tested, as well as a variety of formation temperatures. After mixing, formulations were allowed to stand at room temperature. The results are shown in Table 4.

TABLE 4

| (a) | | | | | | |
|---|---|---|---|---|---|---|
| Lipid w/ Equaconazole | Lipid Conc | Drug Solubility¹ | | | | |
| (30 mg drug/mL) | (mg/mL) | 25° C. | 35° C. | 45° C. | 60° C. | Comment |
| PEG-23 GDL | 200 | + | ++ | ++ | ++ | |
| PEG-12 GDO | 150 | + | ++ | ++ | +++ | |
| PEG-12 GDLO | 150 | + | + | ++ | +++ | |
| PEG-23 GDO | 200 | + | ++ | ++ | ++ | |
| PEG-43 GDO | 200 | − | −/+ | + | ++ | S |
| PEG-12 GDM | 150 | + | ++ | ++ | +++ | |
| PEG-23 GDM | 200 | + | ++ | ++ | ++ | |
| PEG-45 GDM | 200 | − | −/+ | + | + | S |
| PEG-12 GDP | 200 | −/+ | + | ++ | ++ | |
| PEG-23 GDP | 200 | − | −/+ | + | + | S |
| PEG-45 GDP | 200 | − | −/+ | + | + | S |
| PEG-12 GDS | 150 | − | −/+ | + | + | S |
| PEG-23 GDS | 200 | − | + | + | + | S |
| PEG-45 GDS | 200 | − | − | −/+ | + | S |

| (b) | Drug Solubilized @ 40° C. | | | | | |
|---|---|---|---|---|---|---|
| Lipid w/ Equaconazole | | | L/D | L/D | LD ratio = | |
| (30 mg drug/mL) | L/D ratio = 1 | L/D ratio = 3 | ratio = 5 | ratio = 10 | 20 | Comment |
| PEG-23 GDL | − | − | + | + | + | |
| PEG-12 GDO | −/+ | + | ++ | ++ | ++ | |
| PEG-12 GDLO | −/+ | + | + | ++ | +++ | |
| PEG-23 GDO | − | − | + | + | + | |
| PEG-43 GDO | − | − | −/+ | + | + | S |
| PEG-12 GDM | −/+ | + | ++ | ++ | ++ | |
| PEG-23 GDM | − | − | + | + | + | |
| PEG-45 GDM | − | − | −/+ | + | + | S |
| PEG-12 GDP | − | −/+ | + | ++ | ++ | |
| PEG-23 GDP | − | − | + | + | + | S |
| PEG-45 GDP | − | − | + | + | + | S |
| PEG-12 GDS | − | − | + | + | + | S |
| PEG-23 GDS | − | − | + | + | + | S |
| PEG-45 GDS | − | − | −/+ | + | + | S |

### Example 4

### Ketoconazole-Lipid Formulations

Ketoconazole was combined in an aqueous solution with DAG-PEG lipids. A variety of drug-to-lipid ratios were tested, as well as a variety of formation temperatures. After mixing, formulations were allowed to stand at room temperature. The results are shown in Table 5.

TABLE 5

| | | |
|---|---|---|
| (a) | | |
| Lipid w/ Ketoconazole | Lipid Conc | Drug Solubility¹ |

| (20 mg drug/mL) | (mg/mL) | 25° C. | 35° C. | 45° C. | 60° C. | Comment |
|---|---|---|---|---|---|---|
| PEG-23 GDL | 150 | − | − | −/+ | −/+ | S |
| PEG-12 GDO | 150 | + | + | + | ++ | |
| PEG-12 GDLO | 150 | + | + | ++ | +++ | |
| PEG-23 GDO | 200 | − | − | − | −/+ | S |
| PEG-43 GDO | 200 | − | − | −/+ | −/+ | S |
| PEG-12 GDM | 150 | + | + | + | ++ | |
| PEG-23 GDM | 200 | − | − | −/+ | −/+ | S |
| PEG-45 GDM | 200 | − | − | − | − | |
| PEG-12 GDP | 200 | −/+ | + | ++ | ++ | |
| PEG-23 GDP | 200 | − | − | −/+ | −/+ | S |
| PEG-45 GDP | 200 | − | − | − | −/+ | S |
| PEG-12 GDS | 200 | − | − | + | + | S |
| PEG-23 GDS | 200 | − | − | −/+ | + | S |
| PEG-45 GDS | 200 | − | − | − | −/+ | S |

| (b) | Drug Solubilized @ 40° C. | | | | | |
|---|---|---|---|---|---|---|
| Lipid w/ Ketoconazole | | | L/D | L/D | LD ratio = | |
| (20 mg drug/mL) | L/D ratio = 1 | L/D ratio = 3 | ratio = 5 | ratio = 10 | 20 | Comment |
| PEG-23 GDL | − | − | − | −/+ | + | S |
| PEG-12 GDO | − | −/+ | + | ++ | ++ | |
| PEG-12 GDLO | − | −/+ | + | ++ | +++ | |
| PEG-23 GDO | − | − | − | −/+ | + | S |
| PEG-43 GDO | − | − | − | − | + | S |
| PEG-12 GDM | − | −/+ | + | + | + | |
| PEG-23 GDM | − | − | − | −/+ | + | S |
| PEG-45 GDM | − | − | − | − | + | S |
| PEG-12 GDP | − | −/+ | + | ++ | ++ | |
| PEG-23 GDP | − | − | − | −/+ | + | S |
| PEG-45 GDP | − | − | − | − | + | S |
| PEG-12 GDS | − | − | − | + | + | S |
| PEG-23 GDS | − | − | − | −/+ | + | S |
| PEG-45 GDS | − | − | − | − | + | S |

### Example 5

### Antifungal Oral Solution

PEG lipid was added to a vessel equipped with a mixer propeller. The drug substance was added with constant mixing. Mixing continued until the drug was visually dispersed in the lipids. Pre-dissolved excipients were slowly added to the vessel with adequate mixing. Mixing continued until fully a homogenous solution was achieved. A sample formulation is described in Table 6.

TABLE 6

| Ingredient | mg/mL |
|---|---|
| Antifungal Active | 30.0 |
| PEG Lipid | 180 |
| Sodium Citrate, Dihydrate | 0.6 |
| Citric Acid, Monohydrate | 1.5 |
| Sodium Hydroxide | See below |
| Hydrochloric Acid | See below |
| Sodium Benzoate | 2.0 |
| Artificial Flavor | 5.0 |
| Purified Water | qs 1 mL |

The drug may be itraconazole, posaconazole or equaconazole. The lipid may be PEG-12 GDO, or PEG-12 GDM, PEG 12 GDLO or PEG-12 GDP or any combination thereof. Sodium hydroxide is used to prepare a 10% w/w solution in purified water. The targeted pH is in a range of 4.0 to 7.0. HCl is used to adjust pH if necessary.

### Example 6

### Antifungal IV Injectable Solution

The IV solution was prepared as in Example 5, except that the targeted pH range was between 3.5 and 7.0. A sample formulation is described in Table 7.

TABLE 7

| Ingredient | mg/mL |
|---|---|
| Antifungal Active | 30.0 |
| PEG Lipid | 180 |
| Sodium Hydroxide | See Below |
| Phosphoric Acid | 0.02 |
| Purified Water | qs 1 mL |

The drug may be itraconazole, posaconazole or equaconazole. The lipid may be PEG-12 GDO, PEG-12 GDM, PEG-12 GDLO or PEG-12 GDP or any combination thereof. Sodium hydroxide is used to prepare a 10% w/w solution in purified water. The targeted pH is in a range of 3.5 to 7.0. HCl is used to adjust pH if necessary.

### Example 7

### Antifungal Powder for IV Administration

PEG lipids were weighed into a beaker supplied with a mixer. The agitation speed was set to 300±100 RPM. Foaming was avoided. Drug was added, keeping a constant mixing rate. The mixture was then homogenized at 850-900 RPM until the drug substance was visually dispersed in the lipids. Premixed buffer solution was then added at about 300 RPM, and the mixture was homogenized again at about 850 rpm. Finally, the mixture was spray dried using a suitable spray dryer, e.g. Niro Spray Dryer. A sample formulation is described in Table 8.

TABLE 8

| Ingredient | mg/vial |
|---|---|
| Antifungal Active | 50 |
| PEG Lipid | 300 |
| Sodium Hydroxide | See below |
| Phosphoric Acid | See below |
| Sucrose | 100 |
| Purified Water | See below |
| Vial Fill Weights (x = 5 to 20) | 450 x |

The drug may be Itraconazole or Posaconazole or Equaconazole. The lipid may be PEG-12 GDO, PEG-12 GDM, PEG-12 GDLO or PEG-12 GDP or any combination thereof. Sodium hydroxide is used to prepare a 10% w/w solution in purified water. The targeted pH is in a range of 4.0 to 7.0. Phosphoric acid is used to adjust pH if necessary. Water may be added during mixing and then removed by subsequent steps.

### Example 8

### Antifungal Capsules for Oral Administration

Capsules were prepared by first weighing out the required amount of lipids into a beaker, with agitation supplied by a mixer. The agitation speed was set to 300±100 RPM. Foaming was avoided. The drug was drug added with constant mixing. When agglomerates were no longer observed, the mixture was homogenized at about 850-900 RPM until the drug substance was visually dispersed in the lipids. Purified water was added and homogenization repeated. The mixture was pray dried using a suitable spray dryer, e.g., Niro Spray Dryer. The mixture was dried further using a vacuum oven at 65° C.±10° C. until the moisture level was below 1.0%. The mixture was blended for 5 to ten minutes. Excipients were passed through a No. 30 mesh screen and added. Blending was repeated. The mixture was compacted using a Vector Freund Compactor and then screened through a No. 30 mesh screen. Blending was repeated. Mixture was filled into No. 2, two piece hard gelatin capsules using a Minicap capsule filling machine. Capsules were polished using a Key capsule polisher CP-300. A sample formulation is described in Table 9.

TABLE 9

| Ingredient | Mg/capsule |
|---|---|
| Antifungal Active | 50.0 |
| PEG Lipid | 300 |
| Microcrystalline Cellulose NF | 50 |
| Poloxamer 188 NF | 25 |
| Povidone USP | 12.5 |
| Croscarmellose Sodium NF | 12.5 |
| Purified Water | See below |
| Capsule Fill Weights | 450 |

The drug may be itraconazole, posaconazole or equaconazole. The lipid may be PEG-12 GDO, PEG-12 GDM, PEG-12 GDLO or PEG-12 GDP or any mixture thereof. Water may be added during mixing and then removed by subsequent steps.

### Example 9

### Bioavailability of Posaconazole/Itraconazole Formulations

Groups of three male mice (B6D2F1) were used for the studies. Pharmacokinetics (PK) were performed on heparinized mouse plasma samples obtained typically at 0 hr, 0.08 hr, 0.25 hr, 0.5 hr, 1 hr, 2 hr, 4 hr, 8 hr, 16 hr, 24 hr, 31 hr and 48 hr after the bolus IV injection or oral feeding for Posaconazole and at 0 hr, 0.08 hr, 0.25 hr, 0.5 hr, 1 hr, 2 hr, 4 hr, 8 hr, 16 hr and 24 hr for Itraconazole, respectively. Samples were analyzed using a HPLC-MS method. To determine the level of each drug, the drug was first isolated from plasma with a sample pre-treatment. Acetonitrile were used to remove proteins in samples. An isocratic HPLC-MS method was then used to separate the drugs from any potential interference. Drug levels were measured by MS detection with a multiple reaction monitoring (MRM) mode. PK data was analyzed using the WinNonlin program (ver. 5.2, Pharsight) compartmental models of analysis.

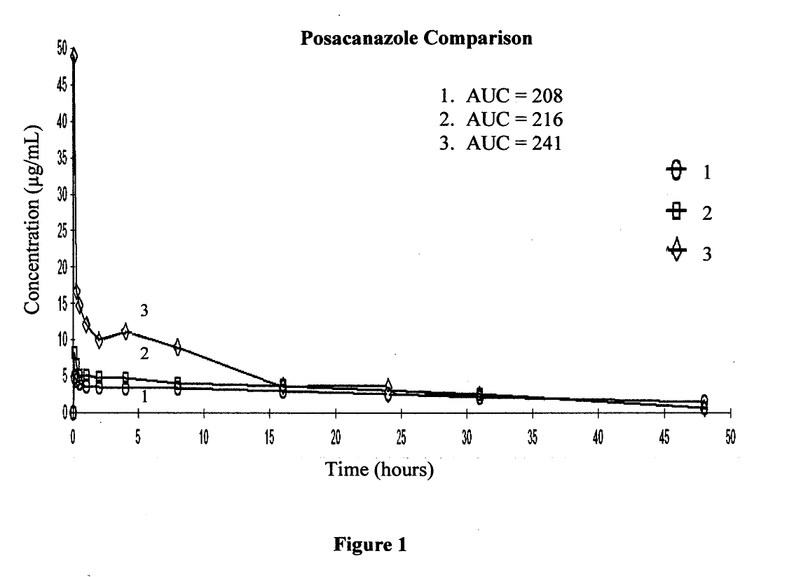

FIG. 1 shows comparison between blood concentrations of various posaconazole formulations administered intravenously. Both a DOPC formulation (1) of posaconazole and (2) a posaconale solution containing 5% dimethyl sulfoxide and 10% Cremophor were cleared more rapidly than a DAG-PEG (PEG-12 GDO) lipid formulation (3) of posaconazole. The dosing strength was 10 mg/kg.

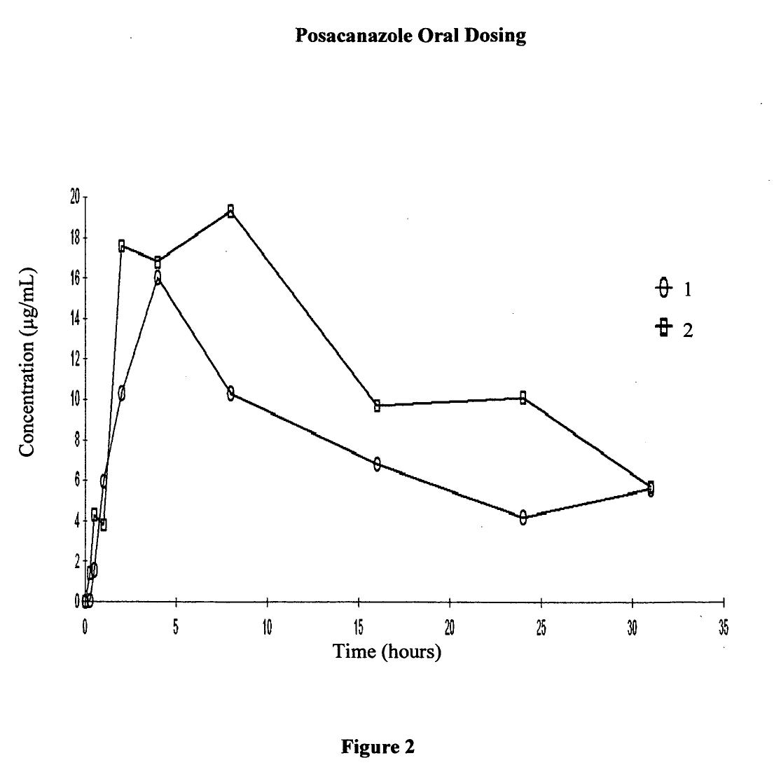

FIG. 2 shows a comparison between blood concentrations of a commercial posaconazole product (1) and a DAG-PEG (PEG-12 GDO) formulation (2) of posaconazole administered orally. The dosing strength was 50/mg/kg. The bioavailability of the DAG-PEG formulation was 45% versus 28% for the commercially available product.

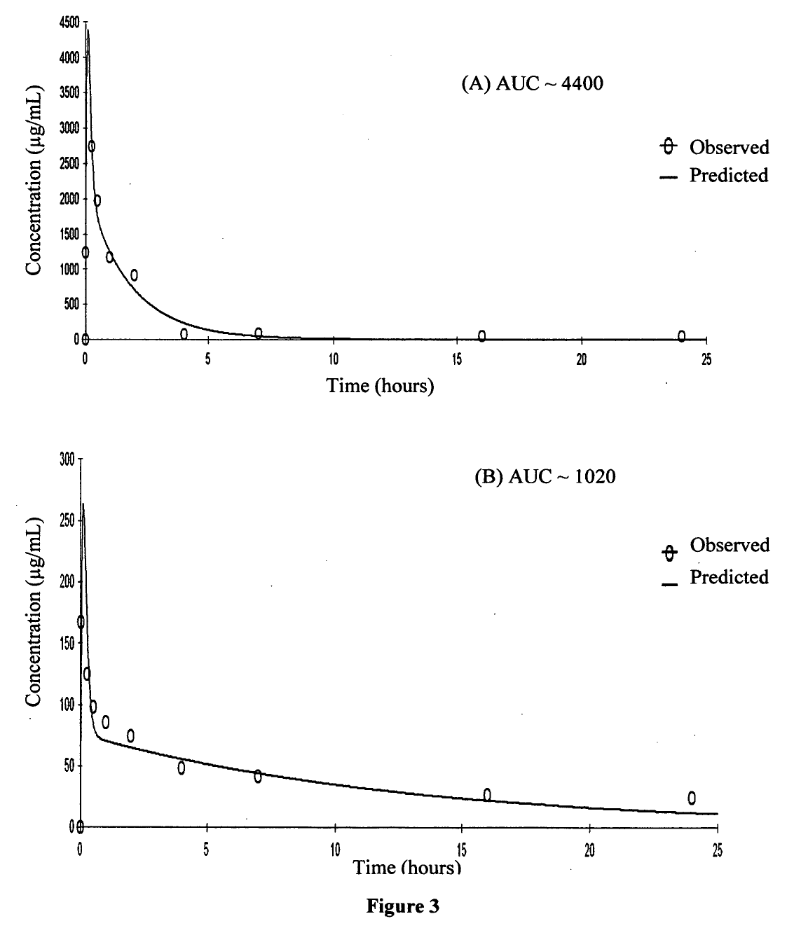

FIG. 3 shows a comparison between (a) a DAG-PEG (PEG-12 GDO) lipid formulation of itraconazole and (b) an aqueous solution of itraconazole containing 5% dimethyl sulfoxide and 10% Cremophor after IV administration. The dosing strength was 20 mg/kg. Area under the curve (AUC) for the DAG-PEG formulation was about 4400 versus 1020 for the nonliposomal formulation.

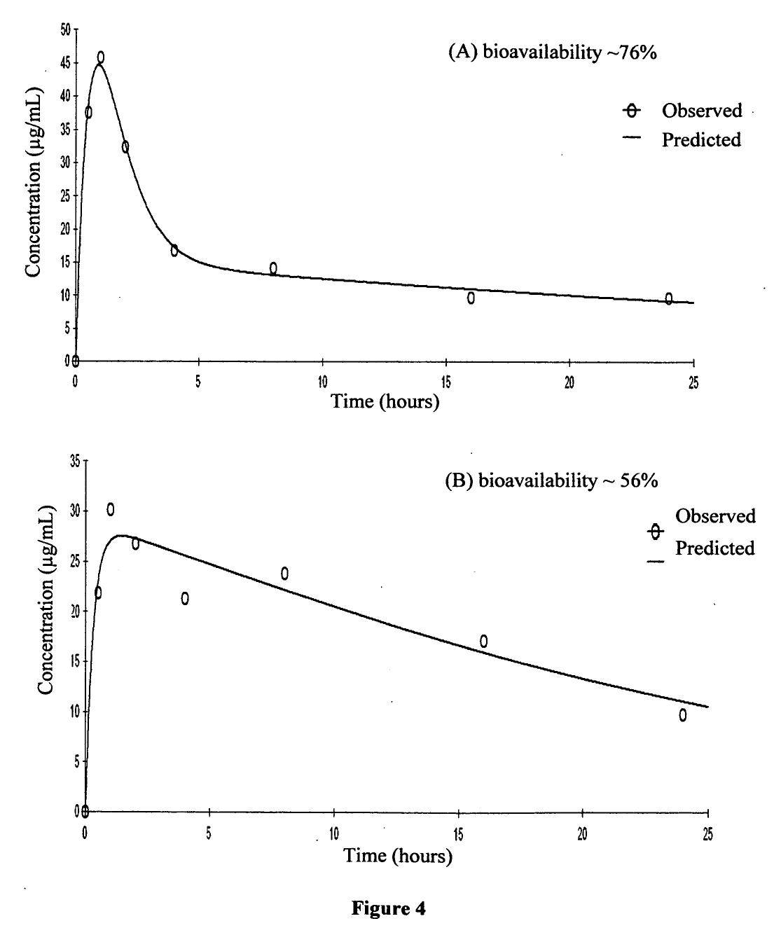

FIG. 4 shows a comparison between oral administrations of (a) DAG-PEG (PEG-12 GDO) lipid formulation of itraconazole and (b) an aqueous solution of itraconazole containing 5% dimethyl sulfoxide and 10% Cremophor. The dosing strength was 20 mg/kg. Bioavailability of the DAG-PEG formulation was about 76% versus about 56% for the nonliposomal formulation.

### Example 10

### Antifungal Topical Cream

PEG lipid was added to a stainless steel vessel equipped with propeller type mixing blades. The drug substance was added with constant mixing. Mixing continued until the drug was visually dispersed in the lipids at a temperature to 60°-65° C. Cholesterol and glycerin were added with mixing. Ethanol and ethyoxydiglycol were added with mixing. Finally Carbopol ETD 2020, purified water and triethylamine were added with mixing. Mixing continued until fully a homogenous cream was achieved. The formulation is described in Table 10.

TABLE 10

| Ingredient | % |
|---|---|
| Antifungal Active | 1.0 |
| PEG Lipid | 5.0 |
| Carbopol ETD 2020 | 0.5 |
| Ethyoxydiglycol | 1.0 |
| Ethanol | 10.0 |
| Glycerin | 1.0 |
| Cholesterol | 0.4 |
| Triethylamine | 0.20 |
| Phosphoric Acid | See below |
| Purified water | qs 100 |

The drug may be ketoconazole, itraconazole, posaconazole or equaconazole. The lipid may be PEG-12 GDS, PEG-23 GDS, PEG-23 GDP or any combination thereof. Phosphoric acid is used to adjust pH if necessary. The targeted pH range was between 3.5 and 7.0.

### Example 11

### Antifungal Topical Solution

The topical solution was prepared as in Example 5, except that active was first dissolved in ethanol and the targeted pH range was between 3.5 and 7.0. A sample formulation is described in Table 11.

TABLE 11

| Ingredient | % |
|---|---|
| Antifungal Active | 1.0 |
| PEG Lipid | 5.0 |
| α-Tocopherol | 0.5 |
| Ethanol | 5.0 |
| Sodium Benzoate | 0.2 |
| Sodium Hydroxide | See Below |
| Phosphoric Acid | See Below |
| Purified Water | qs 100 |

The drug may be ketoconazole, itraconazole, posaconazole or equaconazole. The lipid may be PEG-12 GDO, PEG-12 GDM or PEG-12 GDLO or PEG-12 GDP or any combination thereof. Sodium hydroxide is used to prepare a 10% w/w solution in purified water. Phosphoric acid is used to adjust pH if necessary. The targeted pH is in a range of 3.5 to 7.0.

1. A pharmaceutical composition comprising:
a tetrahydrofuran drug, and a DAG-PEG lipid having a melting temperature below about 25 degrees C. and having packing parameters favorable for the formation of liposomes.

2. The composition of claim 1, where the drug is selected from the group consisting of posaconazole, itraconazole and equaconazole.

3. The composition of claim 2, where the DAG-PEG lipid is selected from the group consisting of PEG-12 GDO, PEG-12 GDLO, PEG-12 GDM and PEG-12 GDP.

4. The composition of claim 2, where the DAG-PEG lipid is a mixture of two or more lipids selected from the group of PEG-12 GDO, PEG-12 GDLO, PEG-12 GDM and PEG-12 GDP.

5. The composition of claim 3, where the drug to lipid ratio is greater than about 1 to 20.

6. The composition of claim 3, where the drug to lipid ratio is greater than about 1 to 5.

7. The composition of claim 5, where the composition further comprises a sugar and is in a dehydrated form.

8. The composition of claim 5, where the composition is a liposomal suspension.

9. The composition of claim 8, where the drug concentration is between about 1 to 50 mg/ml.

10. The composition of claim 5, where the composition further comprises solid excipients and is in the form of a capsule.

11. A method of making a pharmaceutical formulation comprising: selecting a tetrahydrofuran drug; selecting a diacyl PEG lipid having a melting temperature below about 25 degrees C. and having packing parameters favorable for the formation of liposomes; and combining the drug with the lipid in an aqueous solution.

12. The method of claim 11, where the drug is selected from the group consisting of posaconazole, itraconazole and equaconazole.

13. The method of claim 12, where the lipid is selected from the group consisting of
PEG-12 GDO, PEG-12 GDLO, PEG-12 GDM and PEG-12 GDP.

14. The method of claim 13, where the final drug concentration is between about 1 to 50 mg/ml.

15. The method of claim 13, where the final drug to lipid ratio is greater than about 1 to 20.

16. The method of claim 14, where the final drug to lipid ratio is greater than about 1 to 5.
